(19) United States
(12) Patent Application Publication
(10) Pub. No.: US 2006/0074489 A1
(43) Pub. Date: Apr. 6, 2006

(54) Peanut spectacle multi discoid thoraco-lumbar disc prosthesis

(75) Inventor: Vincent Bryan, Quincy, WA (US)

Correspondence Address: HAYNES AND BOONE, LLP, 901 MAIN ST, SUITE 3100, DALLAS, TX, 75202, US

(73) Assignee: SDGI Holdings, Inc., Wilmington, DE (US)

(21) Appl. No.: 11/286,866

(22) Filed: Nov. 23, 2005

Related U.S. Application Data
(63) Continuation of application No. 10/443,422, filed on May 22, 2003.
Application No. 10/443,422 is a continuation of application No. 09/786,073, filed on Jun. 19, 2001, now Pat. No. 6,749,635.
(60) Provisional application No. 60/099,277, filed on Sep. 4, 1998.

(51) Int. Cl. A61F 2/44

(52) U.S. Cl. 623/17.13; 623/17.14

(57) ABSTRACT

A disc prosthesis comprises a first prosthetic device comprising a first upper housing and a first lower housing between which at least one first disc element is movably interposed. The disc prosthesis further comprises a second prosthetic device comprising a second upper housing and a second lower housing between which at least one second disc element is movably interposed. The first and second prosthetic devices are adapted for parallel placement within an intervertebral space.

This application is a continuation of U.S. application Ser. No. 10/443,422, filed May 22, 2003; which is a continuation of U.S. application Ser. No. 09/786,073, filed Jun. 19, 2001; which is a 371 of PCT/US99/20457 filed Sep. 3, 1999; which claims priority to Provisional Application No. 60/099,277, filed Sep. 4, 1998.

This invention relates to the design and use of a unique disc prosthesis for the lumbar and thoracic spine. By placing one or more ovoid resilient prosthetic nuclei in series inside a peanut-shaped housing of metal ceramic or polymeric material, which housing is shaped so that it is separated into two sections longitudinally, a thin profile prosthesis can be created which will allow placement of the device through a small opening for implant into the thoracic or lumbar portion of the spine.

U.S. Pat. No. 5,674,296 is incorporated by reference.

### BACKGROUND

Degenerative disc disease, including disc herniation, may produce disabling symptoms of local pain, radiculopathy or myelopathy in an otherwise clinically stable spine, and may be unresponsive to non-surgical treatment. Several surgical treatments are available to address the symptoms of degenerative disc disease when non-invasive therapies are not effective. These surgical treatments include decompression, discectomy and fusion. These treatments, and in particular the discectomy and fusion procedures, provide relief of clinical symptoms but they do not restore normal or near normal range of motion or cushioning to the affected functional spinal unit (FSU). This can result in acceleration of the degenerative process in spinal discs adjacent to the original surgical operation site. This degenerative process can, in turn, require additional surgical intervention.

Open surgery and endoscopic techniques are often used to provide access to the targeted intervertebral disc space. Posterior, postero-lateral, and anterior approaches allow placement of instrumentation to facilitate exposure of the degenerated disc and the insertion of bone grafts or fusion cages to accomplish bony fusion.

Because of anatomical structure considerations and instrument size restrictions associated with minimally invasive surgical techniques in the anterior lumbar spine, the insertion of a functional disc prosthesis equal in size to the natural disc creates risks due to mechanical interferences with critical vascular structures.

A functional disc prosthesis which provides for a full range of motion of the FSU and for cushioning between two adjacent vertebrae while maintaining stability, intervertebral body spacing and lordosis, is desirable.

More specifically it is an object of the invention to provide a disc prosthesis having a small or narrow profile. The novel exemplary prosthesis has an exterior shape like that of a peanut shell. This peanut shaped housing is comprised of two longitudinally split halves. Each housing half is separated from the other at all times by disk shaped resilient bodies contained therein, and is strong enough to support the loads to which it shall be subjected during the activities of daily living. The discoid nuclei are of smaller diameter than the natural discs they replace, and are positioned in the shell concave interiors of the peanut shaped housing. The housing is configured to accommodate the restrictions imposed by the limited anatomical space available for the surgical placement of the implant, and is small so as to utilize implantation procedures and instrumentation such as those used in an endoscopic procedure.

It is a further object of the invention to provide geometry to engage concave mating surfaces on the vertebral bodies or bones so as to provide proper stability and proper positioning of the opposing engaged vertebrae or vertebral bodies.

Another object is to obviate the need for a second surgical site for bone graft harvesting as may be required when spinal fusion cages are implanted.

And it is a further object of the invention to provide a sheath so as to completely surrounded and enclose the space occupied by the resilient bodies between the two housing halves, thereby restricting the migration of debris outside the prosthesis, restricting cancellous tissue ingrowth into the device, and providing a sealed space around the prosthetic nucleus in which lubricant may be contained.

Still another object of the invention is to provide a disc prosthesis which will permit motion between the housing halves.

A further object of the intention is to provide a disc prosthesis which will provide for cushioning between the housing halves.

It is a still further object of the invention to provide a disc prosthesis which may be used alone or in parallel array with similar prosthesis.

It is yet another object to provide a housing having one or more ports through which a liquid (for example, a saline fluid, hyaluronic acid, or similar lubricating fluid material including for example a hydrogel material) can be introduced into the housing interior space confined within the sheath and partly occupied by the disc for purposes of lubrication, spacing, and/or cushioning. A plug, screw or other can also be provided to seal closed the port following introduction of that material.

Other objects and advantages of the invention will become apparent to those skilled in the art upon reading the following detailed description and upon reference to the drawings. Throughout the drawings, like reference numerals refer to like parts.

### DESCRIPTION OF THE DRAWINGS

FIG. 1 is a top plan view of the novel spinal prosthesis.

FIG. 2 is an end view of the prosthesis shown in FIG. 1.

FIG. 3 is a sectional view taken substantially in the plane of line 3-3 in FIG. 1.

FIG. 4 is an exploded view of the novel prosthesis.

FIG. 5 is a top plan view of the interior of one of the shelves comprising the novel prosthesis.

FIG. 6 is a side elevational view of the shell half shown in FIG. 5.

FIG. 7 is an end view of the shell half shown in FIGS. 5 and 6.

FIG. 8 is a fragmentary view of the shell half shown in FIG. 7, but showing in further detail the half edge shape which is adapted to engage the implant sheath and a circlage wire.

FIG. 9 is an exploded view showing the interiors of the shell halves.

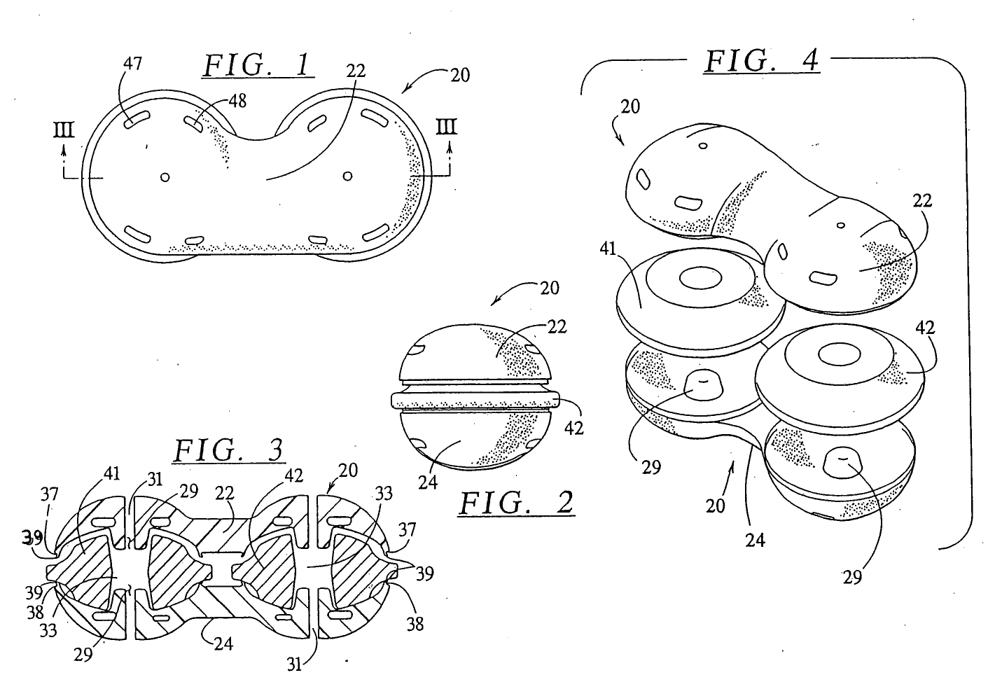

While the invention will be described in connection with a preferred embodiment, it will be understood that it is not intended to limit the invention to this embodiment. On the contrary, it is intended to cover all alternatives, modifications and equivalents as may be included within the spirit and scope of the invention as defined by the appended claims.

To accomplish the objectives set out above, the novel exemplary disc prosthesis 10 includes, as shown in the drawings, a peanut shaped housing 20. The housing 20 includes an upper half housing 22 and a lower half housing 24.

As particularly shown in FIGS. 2, 3, and 4, a plurality of resilient, viscoelastic discs 41, 42 are interposed between the upper half housing 22 and the lower half housing 24 to maintain the housing halves separate from one another and to provide for a defined range of motion between the housing halves and, consequently, for the implant patient's spine. Alternatively, the discs 41, 42 may be made of a suitable hydrogel. The discs can have a relatively soft and resilient interior and a relatively hard and durable exterior. If desired, generally conical bosses or posts 29 can fit into recesses 33 formed in the discs 41, 42 to provide stability and limitation against excessive motion. Also if desired, these posts 29 can be provided with small passageways 31 to permit the introduction of fluids or gel into the interior of the assembled implant. As shown in FIG. 3, circlage wires 37, 38 or other known devices can be fit into grooves 41 (FIG. 8) formed at the edge of the shell halves 22, 24 so as to attach and retained a fluid-retaining sheath 39, as suggested in U.S. Pat. No. 5,674,296.

Ports 31 can be formed in the shell halves 22, 24 to permit lubricating fluids or gels to be introduced into the interior of the assembled implant. The ports 31 can later be sealed by a plug, a screw or the like if desired to prohibit the later expulsion or loss of the introduced fluid or gels. Recesses 47, 48 permit bone ingrowth and consequently firm, permanent attachment of the implant to the mating vertebral bone surfaces.

The prosthetic device 10 can be implanted in the thoracic or lumbar region of the spine through a small surgical opening. One device 20 containing two or more discs 41, 42 may be used, or by placing two such devices 20 in parallel, each containing two or more ovoid discs in series, a full range of motion of the functional spinal unit (FSU) can be achieved. If the discoid material possesses resilient, viscoelastic properties, with the housing being split with the internally placed ovoid discs maintaining the separation of the upper and lower housing members, a cushioning effect may also be realized.

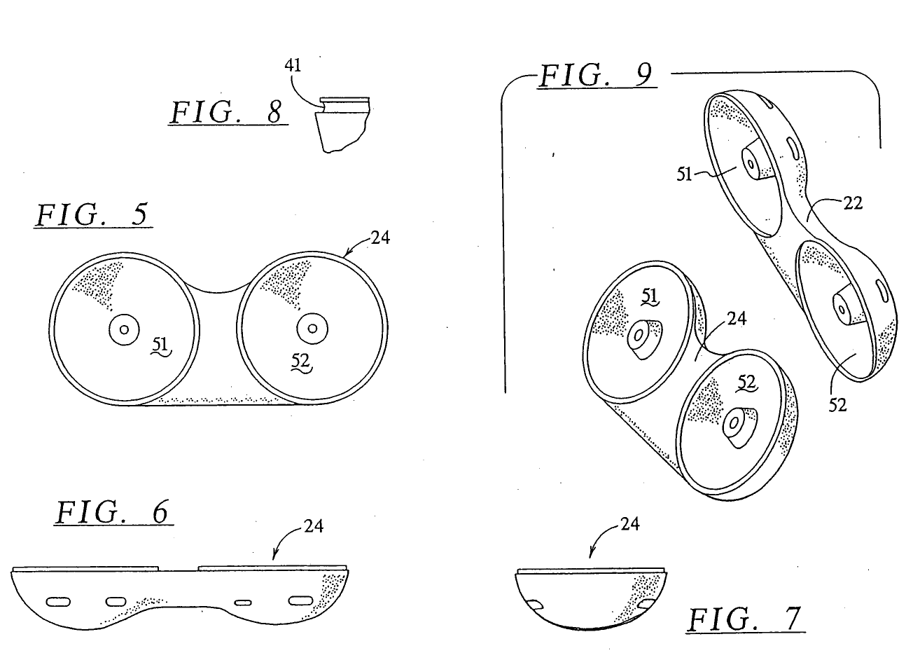

As suggested in FIGS. 5 and 9, each ovoid disc 41, 42 may be partly surrounded and retained by a concave surface 51, 52 formed or contained within the housing, and contoured to accept the upper and lower surface shape of each of the ovoid discs 41, 42 so that the housing 20 comprising the two or more halves or paired shells 23, 24 may slide and/or rotate over the surface of the discs 41, 42 to provide for joint space separation and motion.

The device may be inserted via open or minimally invasive techniques including endoscopy, or by a variety of known surgical anterior, posterior, lateral or other approaches where adequate anatomical space is available. Though the prosthesis is inserted as a single cylindrical unit, its final position is such that one half of the housing is left exclusively in contact with the cephalad vertebral bone with the caudal vertebral bone superior end plate. The discoid vertebral bodies between the cylindrical housing halves contain two or more concave surfaces, allow movement by providing for sliding and rotating in multiple directions and cushioning in response to physiological loads placed upon them.

The following is claimed as invention:

1. A disc prosthesis comprising:
a first prosthetic device comprising a first upper housing and a first lower housing between which at least one first disc element is movably interposed and
a second prosthetic device comprising a second upper housing and a second lower housing between which at least one second disc element is movably interposed,
wherein the first and second prosthetic devices are adapted for parallel placement within an intervertebral space.

2. The disc prosthesis of claim 1 wherein the at least one first disc element has opposite convex surfaces.

3. The disc prosthesis of claim 1 wherein the first prosthetic device further comprises a first post extending from the first upper housing and the at least one first disc element further comprises a recess portion into which the first post is adapted to extend.

4. The disc prosthesis of claim 1 wherein the first and second prosthetic devices are further adapted for separated placement within the intervertebral space.

5. The disc prosthesis of claim 1 wherein the first prosthetic device further comprises a first sheath extending between the first upper and lower housings.

6. The disc prosthesis of claim 5 wherein the first prosthetic device further comprises a circlage retainer for affixing the first sheath the first upper housing.

7. The disc prosthesis of claim 1 wherein the first upper housing comprises a pair of rounded lobes.

8. The disc prostheis of claim 1 wherein the first upper housing comprises a convex surface adapted to slidingly contact the at least one first disc element.

9. The disc prosthesis of claim 8 wherein the first lower housing comprises a convex surface adapted to slidingly contact the at least one first disc element.

10. The disc prosthesis of claim 1 wherein the at least one first disc element is adapted to rotate with respect to the first upper housing.

11. A minimally invasive method of installing a functional spinal unit comprising:
creating at least one posterior opening in a patient;
accessing an intervertebral disc space through the at least one posterior opening;
inserting a first prosthetic device into the intervertebral disc space, wherein the first prosthetic device comprises a first upper housing and a first lower housing between which at least one first disc element is movably interposed; and
inserting a second prosthetic device into the intervertebral disc space and into generally parallel alignment with the first prosthetic device, wherein the second prosthetic device comprises a second upper housing and a second lower housing between which at least one second disc element is movably interposed.

12. The method of claim 11 further comprising engaging the first upper housing with an upper vertebral endplate and engaging the first lower housing with a lower vertebral endplate.

13. The method of claim 12 further comprising engaging the second upper housing with the upper vertebral endplate and engaging the second lower housing with the lower vertebral endplate.

14. The method of claim 11 further comprising inserting the first prosthetic device as a single unit.

15. The method of claim 11 wherein the at least one first disc element comprises a convex upper surface adapted for slidably engaging a convex surface of the first upper housing.

16. The method of claim 15 wherein the at least one first disc element comprises a convex lower surface adapted for slidably engaging a convex surface of the first lower housing.

17. The method of claim 11 wherein the first and second prosthetic devices are inserted into the intervertebral disc space through the at least one posterior opening.

18. The method of claim 11 further comprising limiting motion of the at least one first disc element relative to the first upper and lower housings with a post extending from one of the first housings into a recess in the at least one first disc element.

19. The method of claim 11 wherein the first prosthetic device further comprises a sheath extending between the first upper and lower housings.

20. The method of claim 19 wherein the first prosthetic device further comprises a circlage retainer for affixing the sheath to the first upper housing.
